(19) United States
(12) Patent Application Publication
(10) Pub. No.: US 2023/0241149 A1
(43) Pub. Date: Aug. 3, 2023

(54) TRANSTHYRETIN TETRAMER STABILIZING AGENT, AND PREVENTING AGENT OR PROGRESSION SUPPRESSING AGENT FOR TRANSTHYRETIN AMYLOIDOSIS

(71) Applicants: KANEKA CORPORATION, Osaka (JP); EDUCATIONAL FOUNDATION KYUSHU BUNKA GAKUEN, Sasebo-shi (JP); NATIONAL UNIVERSITY CORPORATION KUMAMOTO UNIVERSITY, Kumamoto-shi (JP)

(72) Inventors: Jinko SAWASHITA, Osaka-shi (JP); Yukio ANDO, Sasebo-shi (JP); Hiroaki MATSUSHITA, Sasebo-shi (JP); Mitsuharu UEDA, Kumamoto-shi (JP); Teruaki MASUDA, Kumamoto-shi (JP); Yohei MISUMI, Kumamoto-shi (JP)

(73) Assignees: KANEKA CORPORATION, Osaka (JP); EDUCATIONAL FOUNDATION KYUSHU BUNKA GAKUEN, Sasebo-shi (JP); NATIONAL UNIVERSITY CORPORATION KUMAMOTO UNIVERSITY, Kumamoto-shi (JP)

(21) Appl. No.: 18/131,450

(22) Filed: Apr. 6, 2023

Related U.S. Application Data
(63) Continuation of application No. PCT/JP2021/037039, filed on Oct. 6, 2021.

(30) Foreign Application Priority Data
Oct. 7, 2020 (JP) 2020-169831

(51) Int. Cl. A61K 36/484; A61K 31/352

(52) U.S. Cl. CPC A61K 36/484; A61K 31/352

(57) ABSTRACT

Provided are a transthyretin tetramer stabilizing agent and a preventing agent or progression suppressing agent for transthyretin amyloidosis. The present invention relates to a transthyretin tetramer stabilizing agent including a hydrophobic licorice extract containing a licorice glabra polyphenol, and to a preventing agent or progression suppressing agent for transthyretin amyloidosis including a hydrophobic licorice extract containing a licorice glabra polyphenol.

### CROSS-REFERENCE TO RELATED APPLICATIONS

This application is a Continuation of PCT International Application No. PCT/JP2021/037039, filed on Oct. 6, 2021, which claims priority under 35 U.S.C. § 119(a) to Patent Application No. 2020-169831, filed in Japan on Oct. 7, 2020, all of which are hereby expressly incorporated by reference into the present application.

### Technical Field

The present invention relates first to a transthyretin tetramer stabilizing agent for stabilizing transthyretin tetramer.

The present invention relates secondly to a transthyretin amyloidosis preventing agent or progression suppressing agent for treating, preventing, or suppressing the progression of, transthyretin amyloidosis in which amyloid fibrils formed by the abnormal aggregation of transthyretin are deposited.

### Background Art

Transthyretin is a β-sheet-rich homotetramer protein in which one subunit is constituted by 127 amino acid residues. It is known that transthyretin has a function by which to bind to and transport retinol (vitamin A) and thyroxine (T4) in the blood and the cerebrospinal fluid. A transthyretin tetramer has two thyroxine binding sites (T4 binding sites) in the dimer-dimer link (Non-Patent Literature 1).

Transthyretin tetramer is usually stable under physiological conditions, but is destabilized by factors, such as aging or genetic abnormality, and thus dissociated into monomers, which in turn undergo misfolding to thereby become aggregates of amyloidogenic intermediates, with the result that the aggregates are formed into amyloids, thus causing the deposition of amyloid fibrils and inducing clinical symptoms of amyloidosis. Examples of known transthyretin amyloidoses include: familial amyloid polyneuropathy (FAP) in which variant transthyretins caused by gene mutation (for example, V30M variant transthyretin) are formed into amyloids, which are deposited in various organs in the whole body, thus inducing organopathy; and senile systemic amyloidosis (SSA) in which wild-type transthyretins free from gene mutation are formed into amyloids through aging, and deposited in the heart, tendon, and the like, inducing dysfunction.

The stabilization of a transthyretin tetramer and suppression of amyloid formation can be achieved with a low-molecular-weight compound that binds to a T4 binding site of transthyretin. Such a compound has led to the development of various pharmaceutical products, which are, however, pharmaceutical products that cause a concern about a serious side effect, and pharmaceutical products that impose an extremely large burden on the national treasury because one dose is extremely expensive, although the disease is designated as intractable, hence minimizing the burden of medical expenses to be paid personally by a patient. In addition, transthyretin amyloidosis is a disease that is not only difficult to cure completely but also develops with a high probability along with aging, and accordingly, is attracting attention to develop preventive measures.

Non-Patent Literature 1 and Non-Patent Literature 2 state that two molecules of glabridin, which is one of licorice glabra polyphenols, have the effect of binding to and stabilizing a transthyretin tetramer, thus alleviating the formation of amyloid fibrils.

Licorice is a plant belonging to Fabaceae Glycyrrhiza and widely distributed in China, Europe, Russia, Afghanistan, Iran, Pakistan, and the like, and is a plant having a long history of ingestion in which the root and the like of the plant have been utilized as food and herbal medicine.

### CITATION LIST

### Non-Patent Literature

Non-Patent Literature 1: Yokoyama, T. et al., Biol. Pharm. Bull. 41, 979-984 (2018)

Non-Patent Literature 2: Yokoyama, T. et al., J. Med. Chem. 57, 1090-1096 (2014)

### SUMMARY OF INVENTION

### Technical Problem

The present invention is directed to preventing, treating or suppressing the progression of, transthyretin amyloidosis, for which the stabilization of a transthyretin tetramer is effective. Hence, the invention makes it possible to develop a stabilizing preparation for a transthyretin tetramer, and to administer the preparation to a patient before disease onset, thus making it possible to prevent transthyretin amyloidosis. In view of this, an object of the present invention is to provide a transthyretin tetramer stabilizing agent, and a preventing agent or progression suppressing agent for transthyretin amyloidosis.

### Solution to Problem

As above-mentioned, Non-Patent Literature 1 and Non-Patent Literature 2 describe glabridin as singularly contributing to the stabilization of a transthyretin tetramer, but no study has conventionally been made on the effect that a hydrophobic licorice extract containing another component in addition to glabridin has for the stabilization of a transthyretin tetramer.

The present inventors have made intensive studies, and as a result, have come to complete the following invention through the discovery that a hydrophobic licorice extract containing a licorice glabra polyphenol is effective particularly as an active component of a transthyretin tetramer stabilizing agent and an active component of a preventing agent, treatment agent or progression suppressing agent for transthyretin amyloidosis.

Thus, the present invention is directed to:

(1) A transthyretin tetramer stabilizing agent including a hydrophobic licorice extract containing a licorice glabra polyphenol.

(2) The agent according to (1), wherein the licorice glabra polyphenol contains at least glabrene, glabridin, glabrol, and 4′-O-methylglabridin.

(3) The agent according to (1) or (2), wherein the hydrophobic licorice extract exhibits any one or more of the characteristics b), c), and d) under the following conditions a) in an HPLC analysis:

a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;

b) a ratio of a glabrene peak intensity to a glabridin peak intensity is 38% or more and 41% or less;

c) a ratio of a glabrol peak intensity to a glabridin peak intensity is 44% or more and 47% or less; and

d) a ratio of a 4′-O-methylglabridin intensity to a glabridin peak intensity is 15% or more and 20% or less.

(4) The agent according to any one of (1) to (3), further including, in addition to the hydrophobic licorice extract, any one or more selected from the group consisting of glabrene, glabridin, glabrol, or 4′-O-methylglabridin.

(5) The agent according to any one of (1) to (4), wherein the content of glycyrrhizic acid is 0.005 wt % or less.

(6) A preventing agent or progression suppressing agent for transthyretin amyloidosis including a hydrophobic licorice extract containing a licorice glabra polyphenol.

(7) The agent according to (6), wherein the transthyretin amyloidosis is senile systemic amyloidosis or familial amyloid polyneuropathy.

(8) The agent according to (6) or (7), wherein the licorice glabra polyphenol contains at least glabrene, glabridin, glabrol, and 4′-O-methylglabridin.

(9) The agent according to any one of (6) to (8), wherein the hydrophobic licorice extract exhibits any one or more of the characteristics b), c), and d) under the following conditions a) in an HPLC analysis:

a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;

b) a ratio of a glabrene peak intensity to a glabridin peak intensity is 38% or more and 41% or less;

c) a ratio of a glabrol peak intensity to a glabridin peak intensity is 44% or more and 47% or less; and

d) a ratio of a 4′-O-methylglabridin intensity to a glabridin peak intensity is 15% or more and 20% or less.

(10) The agent according to any one of (6) to (9), further including, in addition to the hydrophobic licorice extract, any one or more selected from the group consisting of glabrene, glabridin, glabrol, or 4′-O-methylglabridin.

(11) The agent according to any one of (1) to (10), in the form of a tablet, a capsule, a granule, or a powder.

(12) The agent according to any one of (1) to (11), including the glabridin in an amount of 4 mg to 1200 mg per one ingestion or dosage unit.

(13) Use of a hydrophobic licorice extract containing a licorice glabra polyphenol, for the manufacture of a composition for stabilizing a transthyretin tetramer.

Here, the composition is preferably in the form of a tablet, a capsule, a granule, or a powder. In addition, the composition preferably contains glabridin in an amount of 4 mg to 1200 mg per one ingestion or dosage unit.

(14) Use of a hydrophobic licorice extract containing a licorice glabra polyphenol, for the manufacture of a pharmaceutical for stabilizing a transthyretin tetramer.

Here, the pharmaceutical is preferably in the form of a preparation for oral administration, such as a tablet, a capsule, a granule, or a powder. In addition, the pharmaceutical preferably contains glabridin in an amount of 4 mg to 1200 mg per one ingestion or dosage unit.

(15) A method of stabilizing a transthyretin tetramer ex vivo, including:

bringing a hydrophobic licorice extract containing a licorice glabra polyphenol in contact with the transthyretin tetramer present ex vivo; and

suppressing the monomerization of the transthyretin tetramer and/or the formation of amyloid fibrils derived from the transthyretin tetramer.

(16) A method of stabilizing a transthyretin tetramer in a subject, including:

administering a hydrophobic licorice extract containing a licorice glabra polyphenol to a subject in need of the stabilization of the transthyretin tetramer; and

suppressing, in the subject, the monomerization of the transthyretin tetramer and/or the formation of amyloid fibrils derived from the transthyretin tetramer.

Here, in the administration, a composition containing the hydrophobic licorice extract and prepared in the form of a preparation for oral administration, such as a tablet, a capsule, a granule, or a powder, is preferably orally administered to the subject. In addition, in the administration, the hydrophobic licorice extract is preferably administered in such a manner that the total amount of the licorice glabra polyphenol per day is 0.01 to 100 mg/kg of body weight, preferably 0.1 to 30 mg/kg of body weight.

(17) A hydrophobic licorice extract including a licorice glabra polyphenol, for stabilizing a transthyretin tetramer ex vivo or in vivo.

(18) The use according to (13), the use according to (14), the method according to (15), the method according to (16), or the hydrophobic licorice extract according to (17), wherein the licorice glabra polyphenol contains at least glabrene, glabridin, glabrol, and 4′-O-methylglabridin.

(19) The use according to (13), the use according to (14), the method according to (15), the method according to (16), or the hydrophobic licorice extract according to (17), wherein the hydrophobic licorice extract exhibits any one or more of the characteristics b), c), and d) under the following conditions a) in an HPLC analysis:

a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;

b) a ratio of a glabrene peak intensity to a glabridin peak intensity is 38% or more and 41% or less;

c) a ratio of a glabrol peak intensity to a glabridin peak intensity is 44% or more and 47% or less; and

d) a ratio of a 4′-O-methylglabridin intensity to a glabridin peak intensity is 15% or more and 20% or less.

(20) The use according to (13), the use according to (14), the method according to (15), the method according to (16), or the hydrophobic licorice extract according to (17), wherein the hydrophobic licorice extract is used in combination with any one or more selected from the group consisting of glabrene, glabridin, glabrol, or 4′-O-methylglabridin.

(21) The use according to (13), the use according to (14), the method according to (15), the method according to (16), or the hydrophobic licorice extract according to (17), wherein the amount of glycyrrhizic acid contained in the hydrophobic licorice extract is 0.005 wt % or less.

(22) Use of a hydrophobic licorice extract containing a licorice glabra polyphenol, for the manufacture of a composition for preventing, or suppressing the progression of, transthyretin amyloidosis.

Here, the composition is preferably in the form of a tablet, a capsule, a granule, or a powder. In addition, the composition preferably contains glabridin in an amount of 4 mg to 1200 mg per one ingestion or dosage unit.

(23) Use of a hydrophobic licorice extract containing a licorice glabra polyphenol, for the manufacture of a pharmaceutical for preventing, or suppressing the progression of, transthyretin amyloidosis.

Here, the pharmaceutical is preferably in the form of a preparation for oral administration, such as a tablet, a capsule, a granule, or a powder. In addition, the pharmaceutical preferably contains glabridin in an amount of 4 mg to 1200 mg per one ingestion or dosage unit.

(24) A method of treating, preventing, or suppressing the progression of, transthyretin amyloidosis in a subject, including:

administering a hydrophobic licorice extract containing a licorice glabra polyphenol to a subject in need of the prevention of, or the suppression of progression of, transthyretin amyloidosis; and

suppressing, in the subject, the monomerization of a transthyretin tetramer and/or the formation of amyloid fibrils derived from the transthyretin tetramer.

Here, in the administration, a composition containing the hydrophobic licorice extract and prepared in the form of a preparation for oral administration, such as a tablet, a capsule, a granule, or a powder, is preferably orally administered to the subject. In addition, in the administration, the hydrophobic licorice extract is preferably administered in such a manner that the total amount of the licorice glabra polyphenol per day is 0.01 to 100 mg/kg of body weight, preferably 0.1 to 30 mg/kg of body weight.

(25) A hydrophobic licorice extract including a licorice glabra polyphenol, for preventing, or suppressing the progression of, transthyretin amyloidosis.

(26) The use according to (22), the use according to (23), the method according to (24), or the hydrophobic licorice extract according to (25), wherein the transthyretin amyloidosis is senile systemic amyloidosis or familial amyloid polyneuropathy.

(27) The use according to (22), the use according to (23), the method according to (24), or the hydrophobic licorice extract according to (25), wherein the licorice glabra polyphenol contains at least glabrene, glabridin, glabrol, and 4′-O-methylglabridin.

(28) The use according to (22), the use according to (23), the method according to (24), or the hydrophobic licorice extract according to (25), wherein the hydrophobic licorice extract exhibits any one or more of the characteristics b), c), and d) under the following conditions a) in an HPLC analysis:

a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;

b) a ratio of a glabrene peak intensity to a glabridin peak intensity is 38% or more and 41% or less;

c) a ratio of a glabrol peak intensity to a glabridin peak intensity is 44% or more and 47% or less; and

d) a ratio of a 4′-O-methylglabridin intensity to a glabridin peak intensity is 15% or more and 20% or less.

(29) The use according to (22), the use according to (23), the method according to (24), or the hydrophobic licorice extract according to (25), wherein the hydrophobic licorice extract is used in combination with any one or more selected from the group consisting of glabrene, glabridin, glabrol, or 4′-O-methylglabridin.

(30) The use according to (22), the use according to (23), the method according to (24), or the hydrophobic licorice extract according to (25), wherein the amount of glycyrrhizic acid contained in the hydrophobic licorice extract is 0.005 wt % or less.

The present description includes the contents disclosed in Japanese Patent Application No. 2020-169831, which is a priority document of the present application.

### Advantageous Effects of Invention

A transthyretin tetramer stabilizing agent according to the present invention makes it possible to maintain transthyretin tetramer stably, and makes it possible to suppress the monomerization of the tetramer and the formation of amyloid fibrils.

A preventing agent, treatment agent or progression suppressing agent for transthyretin amyloidosis according to the present invention can prevent or suppress transthyretin amyloidosis, such as familial amyloid polyneuropathy (FAP) or senile systemic amyloidosis (SSA).

### BRIEF DESCRIPTION OF DRAWINGS

FIG. 1 shows the ratios of the transthyretin monomers to the tetramers (monomer/tetramer) in serums in Test Example 1 in Example 4, in which the serums had been added with a medium-chain fatty acid triglyceride solution containing a hydrophobic licorice extract and with glabridin in such a manner that the amounts of the glabridin were 0, 10, and 50 μM respectively, and the resulting serums had been incubated. The white circles represent the ratios in the serums added with the glabridin. The black circles represent the ratios in the serums added with the medium-chain fatty acid triglyceride solution containing a hydrophobic licorice extract. A lower ratio demonstrates that the stability of the transthyretin tetramer is higher.

FIG. 2 shows the ratios of the transthyretin monomers to the tetramers (monomer/tetramer) in plasma in Test Example 2 in Example 4, in which a medium-chain fatty acid triglyceride solution containing a hydrophobic licorice extract had been ingested to humans over a period of 12 weeks (the amount of ingestion was 300 mg/day), from whom the plasma was obtained at time intervals. A lower ratio demonstrates that the stability of the transthyretin tetramer is higher.

### DESCRIPTION OF EMBODIMENTS

### Hydrophobic Licorice Extract

In the present invention, a licorice to be used as a raw material for an extract is subject to no particular limitation as long as the licorice is a plant belonging to Glycyrrhiza. Specific examples of licorices include Glycyrrhiza uralensis (G. uralensis), Glycyrrhiza inflata (G. inflata), Glycyrrhiza glabra (G. glabra), Glycyrrhiza eurycarpa (G. eurycarpa), Glycyrrhiza aspera (G. aspera), and the like. Preferable examples include G. uralensis, G. inflata, G. glabra, and the like. Still more preferable examples include G. glabra.

The licorice glabra polyphenol is subject to no particular limitation as long as the licorice glabra polyphenol is a polyphenol component contained in such a licorice as above-mentioned. Specific examples of the licorice glabra polyphenol include glabrene, glabridin, glabrol, 4′-O-methylglabridin, glycycoumarin, glycyrol, glycyrin, liquiritigenin, glicoricone, 3′-hydroxy-4′-O-methylglabridin, glyurallin B, licocoumarone, gancaonin I, dehydroglyasperin D, echinatin, isolicoflavonol, dehydroglyasperin C, glyasperin B, glycyrrhisoflavanone, lupiwighteone, glyasperin D, semilicoisoflavone B, and the like.

Many of the licorice glabra polyphenols belong to a group of compounds classified into prenylflavonoids, and have a structure in which one or more C5 isoprene units are bound to diphenyl propane.

A hydrophobic licorice extract to be used in the present invention preferably contains one or more of the above-mentioned compounds as the licorice glabra polyphenol, more preferably contains one or more selected from the group consisting of glabrene, glabridin, glabrol, or 4′-O-methylglabridin, still more preferably contains glabrene, glabridin, glabrol, and 4′-O-methylglabridin.

The licorice glabra polyphenol in the hydrophobic licorice extract may be present in the form of a salt, an ester, a glycoside, or the like. Examples of the salt of the licorice glabra polyphenol include: a salt with an acid acceptable in the final applications, such as pharmaceutical products, food or drink product, and animal feeding stuff, examples of which acid include hydrochloric acid, sulfuric acid, methanesulfonic acid, fumaric acid, maleic acid, succinic acid, acetic acid, benzoic acid, oxalic acid, citric acid, tartaric acid, carbonic acid, or phosphoric acid; a salt with a base acceptable in the final applications, examples of which salt include an alkali metal salt, for example, a sodium or potassium salt; an alkaline earth metal salt, for example, a calcium or magnesium salt; and a salt with a suitable organic ligand, for example, quaternary ammonium. Examples of the ester of the licorice glabra polyphenol include a fatty acid ester, specific examples of which include: an ester with a long-chain fatty acid, such as oleic acid, palmitic acid, stearic acid, linoleic acid, or linolenic acid; and an ester with a short- or medium-chain fatty acid, such as acetic acid or butyric acid. Examples of a glycoside of the licorice glabra polyphenol include a glycoside in which a sugar component, such as a monosaccharide, a disaccharide, a trisaccharide, an oligosaccharide, or a polysaccharide is bound.

In a preferable embodiment, a hydrophobic licorice extract to be used in the present invention preferably exhibits any one or more, more preferably two or more, particularly preferably all, of the characteristics b), c), and d) under the following conditions a) in an HPLC analysis:

a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;

b) a ratio of a glabrene peak intensity (peak area) to a glabridin peak intensity (peak area) is 38% or more and 41% or less;

c) a ratio of a glabrol peak intensity (peak area) to a glabridin peak intensity (peak area) is 44% or more and 47% or less; and

d) a ratio of a 4′-O-methylglabridin intensity (peak area) to a glabridin peak intensity (peak area) is 15% or more and 20% or less.

In the above-mentioned a), the gradient of the mobile phase A and the mobile phase B is a condition in which the ratio of the mobile phase A to the total amount of the mobile phase A and the mobile phase B is preferably constant at 50% (v/v) after the start of analysis until 20 minutes, then increased to 80% (v/v) at a constant ratio after the 20 minutes until 75 minutes, then constant at 100% (v/v) after the 75 minutes until 80 minutes, and constant at 50% (v/v) after the 80 minutes until 100 minutes.

In the above-mentioned a), for example, a YMC J'sphere ODS-H80 (YMC Co., Ltd.) can be used as the ODS column. The size of the ODS column is, for example, 4.6 mm in inner diameter×250 mm in length.

As a measurement method for the amount of the licorice glabra polyphenol, a method that can be used as a measurement method for the amount of a polyphenol can be utilized. Examples of such a measurement method include: a colorimetric method, such as a ferrous tartrate method, a Prussian blue method, a Folin-Ciocalteau method, or a Folin-Denis method; and an HPLC method for component-by-component measurement. Any of the measurement methods may be used. For the measurement of the amount of the whole polyphenol, a Folin-Denis method or a Folin-Ciocalteau method is often used. With a Folin-Denis method or a Folin-Ciocalteau method, using a standard substance makes it possible to prepare a calibration curve based on the standard substance, and to make a measurement in terms of the standard substance. To measure the amount of the licorice glabra polyphenol in the hydrophobic licorice extract or in the composition containing a hydrophobic licorice extract, glabridin can be used as a standard substance, for example, in the case of a Glycyrrhiza⋅glabra (G. glabra) extract. Specifically, for example, using the below-mentioned method described in the Examples section, the amount of the licorice glabra polyphenol component contained in the composition containing a hydrophobic licorice extract can be determined as a value in terms of glabridin.

A part of a plant, when used to obtain a hydrophobic licorice extract, is subject to no particular limitation. The whole plant or any part of a licorice, such as a leaf, stem, root (rhizome), flower, seed, or the like, may be used.

The licorice glabra polyphenol is hydrophobic, and hence, in the present invention, the hydrophobic licorice extract can be used as a licorice extract containing the licorice glabra polyphenol.

The hydrophobic licorice extract containing the licorice glabra polyphenol can be an extract obtained by extracting a hydrophobic component from a licorice. Examples of the extract include: a liquid extract containing an extraction solvent of a licorice; such a concentrate or dried material of the liquid extract as obtained by removing part or all of the extraction solvent from the liquid extract; and a treated material of the liquid extract, the concentrate, or the dried material. Examples of the treated material include a dilution of the liquid extract, the concentrate, or the dried material; and a treated material having the licorice glabra polyphenol concentration increased by concentrating or purifying (or partially purifying) the licorice glabra polyphenol in the liquid extract, the concentrate, or the dried material. A method of obtaining a hydrophobic licorice extract from a licorice is subject to no particular limitation. For example, a hydrophobic component can be obtained from a licorice or a powder thereof, cultured cells of a licorice, or the like by extraction using an organic solvent, or by the like. Alternatively, a hydrophilic component is preliminarily extracted or removed from a licorice using water or an alkaline water solution. Then, from the licorice residue or the residue dried, a hydrophobic component in the licorice can be obtained by extraction using an organic solvent. Alternatively, an extract can be obtained by using another kind of organic solvent to further extract the hydrophobic extract once extracted by the above-mentioned method.

An organic solvent to be used as an extraction solvent is preferably that which is permitted to be used for producing or processing pharmaceutical products, food products, food additives, and the like. Examples of the organic solvent include: organic solvents, such as alcohols (for example, ethanol), esters (for example, ethyl acetate), ketones (for example, acetone), and hydrocarbons (for example, hexane); fats (for example, medium-chain fatty acid triglycerides); and the like. The organic solvent is preferably an alcohol, a ketone, or a fat, specifically preferably ethanol, an acetone medium-chain fatty acid triglyceride, or the like. The organic solvents may be used singularly, or used in mixture of two or more kinds thereof. The extraction solvent is more preferably one or more selected from ethanol, acetone, or a medium-chain fatty acid triglyceride, particularly preferably one or more selected from ethanol or a medium-chain fatty acid triglyceride. Alternatively, such an organic solvent may be used in the form of a hydrous solvent. However, to reduce the amount of the below-mentioned glycyrrhizic acid (glycyrrhizin) to a low level, the extraction solvent preferably has a lower hydrous ratio. The extraction solvent more preferably contains no water.

Specific examples of a more preferable hydrophobic licorice extract containing the licorice glabra polyphenol include an ethanol licorice extract. The ethanol licorice extract may be an ethanol licorice extract obtained by further removing components insoluble in the medium-chain fatty acid triglyceride.

As the hydrophobic licorice extract, the extract extracted using an organic solvent may be used directly or may be used after being partially purified or well purified, for example, by column treatment, deodorization, decolorization, and/or the like in a further purifying step.

The amount of the licorice glabra polyphenol contained in the hydrophobic licorice extract is subject to no particular limitation, and is preferably 50 wt % or more, more preferably 60 wt % or more, still more preferably 70 wt % or more.

### Transthyretin Tetramer Stabilizing Agent

A transthyretin tetramer stabilizing agent according to the present invention has the effect of stabilizing the transthyretin tetramer, and suppressing the monomerization of the transthyretin. Stabilizing the transthyretin tetramer makes it possible to suppress the tetramer from being formed into amyloid. The present inventors have discovered that a hydrophobic licorice extract containing the licorice glabra polyphenol has a markedly higher effect of stabilizing the transthyretin tetramer, compared with glabridin that is conventionally known for achieving the effect of stabilizing the transthyretin tetramer.

The origin of a transthyretin as a subject for stabilization is subject to no particular limitation, and is usually a mammal, preferably a human. The amino acid sequence of a transthyretin may be a wild-type, or may have a mutation.

In one embodiment, the transthyretin tetramer stabilizing agent according to the present invention can be used in applications in which the agent in the form of a pharmaceutical product, food or drink product, or the like is administered to a subject or ingested by a subject, and stabilizes the transthyretin tetramer in vivo, for example, in the blood or the cerebrospinal fluid in the subject.

In addition, another embodiment of the present invention relates to a method of stabilizing a transthyretin tetramer in a subject, including:

administering a hydrophobic licorice extract containing a licorice glabra polyphenol to a subject in need of the stabilization of the transthyretin tetramer; and

suppressing, in the subject, the monomerization of the transthyretin tetramer and/or the formation of amyloid fibrils derived from the transthyretin tetramer.

A subject for the transthyretin tetramer stabilizing agent according to the present invention and the method of stabilizing the transthyretin tetramer according to the present invention is a human or a non-human animal in need of the stabilization of the transthyretin tetramer, preferably a human. Examples of the non-human animal include farm animals, pet animals, competition animals, and the like. Examples of the farm animals include, but are not limited particularly to: domestic animals, such as horses, cattle, pigs, sheep, goats, camels, and llamas; laboratory animals, such as mice, rats, guinea pigs, and rabbits; and domestic fowls, such as chickens, ducks, turkeys, and ostriches. Examples of the pet animals include, but are not limited particularly to, dogs, cats, and the like. Examples of the competition animals include, but are not limited particularly to, race horses and the like. The non-human animal is particularly preferably a mammal.

The frequency of administration of, and the amount of administration of, the transthyretin tetramer stabilizing agent according to the embodiment to a subject can be suitably adjusted in accordance with the age, gender, condition, and the like of the subject. The amount of the hydrophobic licorice extract to be administered per day can be adjusted suitably, and can be, for example, 0.01 to 100 mg/kg of body weight, preferably 0.1 to 30 mg/kg of body weight, as the daily total amount of the licorice glabra polyphenol. The frequency of administration per day can be adjusted suitably, and can be, for example, once or more, twice or more, or five times or less. The above-mentioned examples of the amount of administration and the frequency of administration are particularly preferable in cases where the subject is an adult. The route of administration may be oral administration or parenteral administration, and is preferably oral administration.

In another embodiment, the transthyretin tetramer stabilizing agent according to the present invention is present in an ex vivo sample containing the transthyretin tetramer, and can thus be used in applications in which the transthyretin tetramer is stabilized in the sample. Examples of the sample include blood containing the transthyretin tetramer, a sample derived from a body fluid, such as the cerebrospinal fluid, and a solution containing the transthyretin tetramer.

Yet another embodiment of the present invention relates to a method of stabilizing a transthyretin tetramer ex vivo, including:

bringing a hydrophobic licorice extract containing a licorice glabra polyphenol in contact with the transthyretin tetramer present ex vivo; and

suppressing the monomerization of the transthyretin tetramer and/or the formation in amyloid fibrils from the transthyretin tetramer.

The transthyretin tetramer stabilizing agent can be that which contains a hydrophobic licorice extract containing the licorice glabra polyphenol, and may be a composition further containing one or more other components (for example, a composition or pharmaceutical for stabilizing the transthyretin tetramer). Preferable embodiments of the transthyretin tetramer stabilizing agent will be described below.

### A Preventing Agent or Progression Suppressing Agent for Transthyretin Amyloidosis

A preventing agent or progression suppressing agent for transthyretin amyloidosis according to the present invention is administered to a subject in need of treatment, the prevention of, or the suppression of the progression of, transthyretin amyloidosis, and can stabilize the transthyretin tetramer, and treat, prevent, or suppress the progression of, the formation in amyloid from the transthyretin through the effect of suppressing the monomerization of the transthyretin. The present inventors have discovered that, compared with glabridin that is conventionally known for achieving the effect of stabilizing the transthyretin tetramer, a hydrophobic licorice extract containing the licorice glabra polyphenol has a markedly higher effect of stabilizing the transthyretin tetramer, and accordingly has a markedly higher activity for treating, preventing, or suppressing the progression of, transthyretin amyloidosis.

In addition, another embodiment of the present invention relates to a method of treating, preventing, or suppressing the progression of, transthyretin amyloidosis in a subject, including:

administering a hydrophobic licorice extract containing a licorice glabra polyphenol to a subject in need of the prevention of, or the suppression of progression of, transthyretin amyloidosis; and

suppressing, in the subject, the monomerization of the transthyretin tetramer and/or the formation of an amyloid fibril from the transthyretin tetramer.

In an embodiment, a subject for a preventing agent or progression suppressing agent for transthyretin amyloidosis according to the present invention and for a method of treating, preventing, or suppressing the progression of, transthyretin amyloidosis according to the present invention is a human or a non-human animal in need of the treatment of, prevention of, or the suppression of the progression of, transthyretin amyloidosis, preferably a human. Specific examples of the subject are as above-mentioned in relation to the transthyretin tetramer stabilizing agent.

The frequency of administration of, and the amount of administration of, a treating agent, preventing agent or progression suppressing agent for transthyretin amyloidosis according to the embodiment to a subject can be suitably adjusted in accordance with the age, gender, condition, and the like of the subject. The amount of the hydrophobic licorice extract to be administered per day can be adjusted suitably, and can be, for example, 0.01 to 100 mg/kg of body weight, preferably 0.1 to 30 mg/kg of body weight, as the daily total amount of the licorice glabra polyphenol. The frequency of administration per day can be adjusted suitably, and can be, for example, once or more, twice or more, or five times or less. The above-mentioned examples of the amount of administration and the frequency of administration are particularly preferable in cases where the subject is an adult. The route of administration may be oral administration or parenteral administration, and is preferably oral administration.

The treating agent, preventing agent or progression suppressing agent for transthyretin amyloidosis can be that which contains a hydrophobic licorice extract containing the licorice glabra polyphenol, and may be a composition further containing one or more other components (for example, a composition or pharmaceutical for preventing, or suppressing the progression of, transthyretin amyloidosis). Preferable embodiments of the treating agent, preventing agent or progression suppressing agent for transthyretin amyloidosis will be described below.

Examples of the transthyretin amyloidosis to be treated, prevented or suppressed from progression include senile systemic amyloidosis and familial amyloid polyneuropathy.

### Preferable Embodiments of Transthyretin Tetramer Stabilizing Agent and Preventing Agent or Progression Suppressing Agent for Transthyretin Amyloidosis

Hereinafter, the transthyretin tetramer stabilizing agent and the treating agent, preventing agent or progression suppressing agent for transthyretin amyloidosis are collectively referred to as the “agent according to the present invention”.

The agent according to the present invention can be that which contains a hydrophobic licorice extract containing a licorice glabra polyphenol, may be that which is composed of the hydrophobic licorice extract alone, or may be a composition containing the hydrophobic licorice extract and one or more other components.

The one or more other components can be, for example, one or more components acceptable as food products (ordinary food products, food products for specified health use, food products with function claims, dietary supplements, and the like), pharmaceutical products (pharmaceutical products for humans or pharmaceutical products for non-human animals), quasi-drugs, cosmetics, or animal feeding stuff (domestic animal feeding stuff or pet foods). Examples of the one or more other components acceptable as food products, pharmaceutical products, quasi-drugs, cosmetics, or animal feeding stuff include the below-described components that can be contained in the agent according to the present invention, and are other than the hydrophobic licorice extract.

The agent according to the present invention may further include, in addition to the hydrophobic licorice extract, any one or more polyphenols selected from the group consisting of glabrene, glabridin, glabrol, or 4′-O-methylglabridin. The one or more polyphenols selected from the group consisting of glabrene, glabridin, glabrol, or 4′-O-methylglabridin are blended in after being prepared separately from the above-mentioned hydrophobic licorice extract. The polyphenol prepared separately may be that which is chemically synthesized. The polyphenol prepared separately may be that which has been extracted from a biological sample, such as a plant, a microorganism, or an animal, and, if desired, purified. The polyphenol prepared separately may be produced by fermentation using a microorganism having the capability to produce the polyphenol. The microorganism having the capability to produce the polyphenol may be a genetically engineered microorganism, or may be a wild-type microorganism.

The agent according to the present invention may also include, in addition to the hydrophobic licorice extract, a polyphenol other than glabrene, glabridin, glabrol, and 4′-O-methylglabridin. Examples of the other polyphenol include genistein, daidzein, quercetin, rutin, catechin, epigallocatechin gallate, hesperidin, nobiletin, tyrosol, hydroxy tyrosol, oleuropein, naringenin, caffeic acid, apple polyphenol, tea polyphenol, gallic acid, and the like. The other polyphenol is preferably genistein, daidzein, quercetin, rutin, catechin, epigallocatechin gallate, hesperidin, nobiletin, naringenin, caffeic acid, apple polyphenol, or tea polyphenol. These other polyphenols may be used singularly, or used in mixture of two or more kinds thereof.

The amount of the hydrophobic licorice extract contained in the agent according to the present invention is subject to no particular limitation, and is preferably 0.1 wt % or more, more preferably 1 wt % or more, particularly preferably 3 wt % or more. In addition, the amount of the hydrophobic licorice extract contained in the agent according to the present invention is preferably 0.1 wt % or more, more preferably 1 wt % or more, in terms of the licorice glabra polyphenol content. The upper limit of the amount of the hydrophobic licorice extract contained in the agent according to the present invention is subject to no particular limitation. The more the licorice polyphenol component is contained, the more preferable. The amount is preferably 99 wt % or less, more preferably 90 wt % or less, taking account of containing another effective component(s) in a necessary amount.

In cases where the agent according to the present invention is ingested by a subject, or administered to the subject, the agent according to the present invention contains glabridin preferably in an amount of 0.40 to 4000 mg, more preferably in an amount of 4 mg to 1200 mg, per one ingestion or dosage unit. In cases where the agent according to the present invention is a food product, one ingestion or dosage unit refers to the amount of the food product to be ingested at a time. For example, in cases where the amount of the food product to be ingested at a time is packaged in a bottle or a can in fun-size form, or is packaged individually, one ingestion or dosage unit refers to one packaging unit. In cases where the agent according to the present invention is a pharmaceutical product, one ingestion or dosage unit refers to the recommended amount of the pharmaceutical product to be administered at a time.

In a preferable embodiment of the agent according to the present invention, the amount of glycyrrhizic acid (also referred to as glycyrrhizin) is preferably equal to or less than the amount of the licorice glabra polyphenol contained in the composition on a weight basis, and further preferably equal to or less than the amount of the glabridin. Depending on the extraction condition, the hydrophobic licorice extract can contain a component other than the licorice glabra polyphenol, and, for example, contain glycyrrhizic acid that is a hydrophilic component. For the agent according to the present invention, the amount of the glycyrrhizic acid is preferably smaller from the viewpoint of safety in long-term ingestion or administration. For the agent according to the present invention, it is preferable to use a hydrophobic licorice extract the glycyrrhizic acid content of which is substantially zero or small, for example, 0.005 wt % or less, preferably 0.001 wt % or less.

The agent according to the present invention may further contain a medium-chain fatty acid triglyceride. From the viewpoint of handling, the hydrophobic licorice extract containing the licorice glabra polyphenol is preferably used in the form of a material dissolved in a medium-chain fatty acid triglyceride. In this case, the medium-chain fatty acid triglyceride to be used is subject to no particular limitation as long as the triglyceride is constituted by a C₆₋₁₂ fatty acid. The triglyceride as a main component is preferably constituted by a C₈₋₁₀ saturated fatty acid, more preferably constituted by a C₈ saturated fatty acid. The ratio of the constituent fatty acid in the medium-chain fatty acid triglyceride is subject to no particular limitation. The constituent ratio of the C₈₋₁₀ fatty acid is preferably 50 wt % or more, more preferably 70 wt % or more. In addition, a medium-chain fatty acid triglyceride having a specific gravity of 0.94 to 0.96 at 20° C. and a viscosity of 23 to 28 cP at 20° C. is particularly preferable. These medium-chain fatty acid triglycerides may be naturally-occurring, or may be prepared by transesterification or the like.

In addition, the medium-chain fatty acid triglyceride may be a glycerin fatty acid ester containing a medium-chain fatty acid triglyceride. The medium-chain fatty acid triglyceride is preferably a glycerin fatty acid ester containing a medium-chain fatty acid triglyceride in an amount of 50 wt % or more, more preferably a glycerin fatty acid ester containing a medium-chain fatty acid triglyceride in an amount of 70 wt % or more.

In addition, the agent according to the present invention may further contain a partial glyceride together with the medium-chain fatty acid triglyceride. It is also possible that, in place of the medium-chain fatty acid triglyceride, a partial glyceride of a medium-chain fatty acid is used. The partial glyceride is a glycerin fatty acid ester containing a partial glyceride, preferably a glycerin fatty acid ester containing a partial glyceride in an amount of 50 wt % or more, more preferably a glycerin fatty acid ester containing a partial glyceride in an amount of 70 wt % or more. Here, the partial glyceride is a diglyceride (1,2-diacylglycerol or 1,3-diacylglycerol) or a monoglyceride (1-monoacylglycerol or 2-monoacylglycerol). Any one of these may be used, or a mixture of two or more thereof may be used. A diglyceride is preferable from the viewpoint of processability. In addition, the partial glyceride may be naturally-occurring, or may be prepared by transesterification or the like. A fatty acid residue that constitutes the partial glyceride is, for example, a C₄₋₂₄ fatty acid residue, particularly preferably a C₈₋₁₀ medium-chain fatty acid residue. From among these, a saturated fatty acid, an unsaturated fatty acid, or the like can be selected depending on the application. For example, an unsaturated fatty acid is preferable in cases where fluidity is desired, and a saturated fatty acid is preferable in cases where plasticity is desired. In addition, a branched fatty acid, such as an isostearic acid, can be used.

The agent according to the present invention can contain another component to be used for preparation, besides a hydrophobic licorice extract containing the licorice glabra polyphenol. Examples of the other component to be used for preparation include an excipient, a disintegrator, a lubricant, a binder, an antioxidant, a colorant, an aggregation inhibitor, an absorption enhancer, a solubilizer for an effective component, a stabilizer, a fat, a viscosity modifier, and the like.

Examples of the excipient include, but are not limited particularly to, saccharose, lactose, dextrose, corn starch, mannitol, crystalline cellulose, calcium phosphate, calcium sulfate, magnesium sulfate, and the like.

Examples of the disintegrator include, but are not limited particularly to, starch, agar, calcium citrate, calcium carbonate, sodium hydrogen carbonate, dextrin, crystalline cellulose, carboxy methyl cellulose, tragacanth, and the like.

Examples of the lubricant include, but are not limited particularly to, talc, magnesium stearate, polyethylene glycol, silica, hydrogenated vegetable oil, and the like.

Examples of the binder include, but are not limited particularly to, ethyl cellulose, methyl cellulose, hydroxypropylmethyl cellulose, tragacanth, shellac, gelatin, gum arabic, polyvinylpyrrolidone, polyvinyl alcohol, polyacrylic acid, polymethacrylic acid, sorbitol, and the like.

Examples of the antioxidant include, but are not limited particularly to, ascorbic acid, tocopherol, sodium bisulfate, sodium thiosulfate, sodium pyrosulfite, citric acid, and the like.

Examples of the colorant include, but are not limited particularly to, a colorant permitted to be added to pharmaceutical products, food products, or the like.

Examples of the aggregation inhibitor include, but are not limited particularly to, stearic acid, talc, light anhydrous silicic acid, hydrated silicate dioxide, and the like.

Examples of the absorption enhancer include, but are not limited particularly to, higher alcohols; higher fatty acids; sucrose fatty acid esters; surfactants, such as a sorbitan fatty acid ester, a polyoxyethylene sorbitan fatty acid ester, and a polyglycerin fatty acid ester; and the like.

Examples of the solubilizer for an effective component include, but are not limited particularly to, organic acids, such as fumaric acid, succinic acid, and malic acid, and the like.

Examples of the stabilizer include, but are not limited particularly to, benzoic acid, sodium benzoate, ethyl paraoxybenzoate, propylene glycol, and the like.

Examples of the fat component that can be used include, but are not limited particularly to: vegetable oils, such as corn oil, rapeseed oil, high erucic rapeseed oil, soybean oil, olive oil, safflower oil, cottonseed oil, sunflower oil, rice-bran oil, beefsteak plant oil, perilla oil, linseed oil, evening primrose oil, cacao butter, peanut oil, palm oil, and palm kernel oil; animal oils, such as fish oil, beef tallow, lard, milk fat, and egg yolk oil; fats obtained by separation, hydrogenation, transesterification, or the like using any of these as a raw material; and oil mixtures thereof.

Examples of the viscosity modifier include, but are not limited particularly to, beeswax, Japan tallow, lanolin, microcrystalline wax, liquid paraffin, and the like.

The agent according to the present invention may be, for example, in the form of a food product (an ordinary food product, a food product for specified health use, a food product with function claims, a dietary supplement, or the like), a pharmaceutical product (a pharmaceutical product for humans or a pharmaceutical product for non-human animals), a quasi-drug, a cosmetic, or animal feeding stuff (domestic animal feeding stuff or a pet food), preferably in the form of a food product or a pharmaceutical product.

In cases where the agent according to the present invention is in the form of a food product, a pharmaceutical product, a quasi-drug, animal feeding stuff, or livestock feed, the agent may be in the form of a preparation for oral ingestion. Examples of the preparation for oral ingestion include one in orally ingestible form, such as a tablet, a capsule (a hard capsule, a microcapsule, and a soft capsule), a granule, a powder, a chewable preparation, a syrup, and a liquid. Examples of a capsule base material for the agent to be formed into a capsule include, but are not limited particularly to: gelatins derived from cattle bone, cattle skin, pig skin, fish skin, or the like can to be used. Moreover, examples of other base materials usable as food additives may include: those derived from seaweed, for example, carageenan and alginic acid; those derived from plants or seeds, for example, Locust bean gum and guar gum; those derived from microorganism, for example, pullulan and curdlan; and manufacturing agents which contain celluloses can to be also used.

In addition, the agent according to the present invention may be in the form of a common food product. Examples of the common food product include, but are not limited to: drinks, such as lactic drinks, soft drinks, nutrient drinks, and drinks for beauty; confectionery, such as chewing gums, chocolates, candies, jellies, cakes, biscuits, and crackers; frozen desserts, such as ice creams and ices; noodles, such as udon noodles, Chinese noodles, spaghetti, and instant noodles; paste products, such as boiled fish pastes, chikuwa pastes, and hanpen pastes; seasonings, such as dressings, mayonnaises, and sauces; bread; hams; rice gruel; cooked rice; soups; various kinds of retort pouch food products; various kinds of frozen food products; and the like.

In cases where the agent according to the present invention is in the form that is other than a pharmaceutical product, and is a food product that can be ingested for the maintenance of health, such as a food product for specified health use, a food product with function claims, or a dietary supplement, the agent according to the present invention may be contained in a package, and the package may be labelled as having a function related to stabilizing the transthyretin tetramer and preventing, or suppressing the progression of, transthyretin amyloidosis. Examples of the package include, but are not limit particularly to, boxes, containers, package films, wrapping paper, and the like. In addition, the function labelled on the package may be expressed in a different manner, as long as the function is similar to those above-mentioned.

Furthermore, the agent according to the present invention may be in the form of a parenteral agent. For example, the agent can be in the form of an agent that is applied directly to the skin. In this case, examples of the dosage form include, but are not limited particularly to: an agent (ointment, liniment, lotion, spray agent, or the like) obtained by dissolving or mixing/dispersing the above-mentioned component(s) in a suitable base, and forming the resulting product into a cream, a paste, a jelly, a gel, a milky liquid, or a liquid: an agent (cataplasm or the like) obtained by dissolving or mixing/dispersing the above-mentioned composition(s) in a base, and spreading the resulting product on a support; an agent (plaster, tape, or the like) obtained by dissolving or mixing/dispersing the above-mentioned composition(s) in an adhesive, and spreading the resulting product on a support.

In cases where the agent according to the present invention is in the form of a quasi-drug, the quasi-drug refers to a quasi-drug provided in “Act on Securing Quality, Efficacy and Safety of Products Including Pharmaceuticals and Medical Devices”. Examples of the quasi-drug include oral agents (liquid agents, such as extract agents, elixir agents, syrup agents, tincture agents, and lemonade agents; and solid agents, such as capsules, granules, pills, powders, and tablets) and the like.

### EXAMPLES

### Example 1

A rhizome portion of a licorice (G. glabra) produced in Afghan was used in an amount of 1.0 Kg for extraction with 5.0 L ethanol (at 45° C. for 2 hours for twice), and then allowed to be concentrated under vacuum to obtain 0.45 L of a liquid concentrate. Then, 0.3 L of this liquid concentrate was treated with activated charcoal, and then further concentrated to obtain 123.6 g of an ethanol solution containing a hydrophobic licorice extract (containing 24.8 g of the hydrophobic licorice extract). Furthermore, 50.0 g of this ethanol solution containing a hydrophobic licorice extract was concentrated under vacuum to obtain 10.0 g of the hydrophobic licorice extract.

### Example 2

### Sample Preparation

The ethanol solution containing the hydrophobic licorice extract in Example 1, in an amount of 63.9 g, and 18.8 g of a medium-chain fatty acid triglyceride (Actor M2, manufactured by Riken Vitamin Co., Ltd., with the fatty acid composition of C8:C10=99:1) were mixed, and the resulting mixture was allowed to be concentrated under vacuum, whereby the ethanol was removed. From 28.7 g of the material obtained by the vacuum concentration, the insoluble components were collected by suction filtration, and then, the insoluble components were washed with hexane. The resulting oil collected was added to the previous filtrate. To 26.2 g of the filtrate collected, 4.5 g of a medium-chain fatty acid triglyceride was added to obtain 30.7 g of a medium-chain fatty acid triglyceride solution containing a hydrophobic licorice extract (containing 8.9 g of the hydrophobic licorice extract).

### HPLC Analysis

### Adjustment of HPLC Analysis Sample

In methanol for HPLC, 1 g of the medium-chain fatty acid triglyceride solution containing a hydrophobic licorice extract was dissolved, and the total amount was adjusted to 100 mL.

### HPLC Conditions for Polyphenol Analysis

Column: YMC J'sphere ODS-H80, 4.6 mm in inner diameter×250 mm in length (YMC Co., Ltd.)

Column temperature: 40° C.

Mobile phase A: acetonitrile:methanol (1:1=v/v)

Mobile phase B: 20 mM phosphoric acid water solution

Gradient: the ratio of the mobile phase A to the total amount of the mobile phase A and the mobile phase B was constant at 50% (v/v) after the start of analysis until 20 minutes after the start, then increased to 80% (v/v) at a constant ratio after the 20 minutes until 75 minutes, then constant at 100% (v/v) after the 75 minutes until 80 minutes, and constant at 50% (v/v) after the 80 minutes until 100 minutes.

Flow rate: 1.0 mL/min

Wavelength: UV 282 nm

Amount of sample injected: 20 μL

### Analysis Results

In an HPLC analysis under the above-mentioned conditions in which the sample was a medium-chain fatty acid triglyceride solution containing a hydrophobic licorice extract, the peak intensity of the glabrene was 38.9%, the peak intensity of the glabrol was 45.9%, and the peak intensity of the 4′-O-methylglabridin was 18.7%, in cases where the peak intensity (peak area) of the glabridin was regarded as 100%.

The amount of each component contained in 1 g of the medium-chain fatty acid triglyceride solution containing a hydrophobic licorice extract was as follows: glabrene (4.4 mg), glabridin (30.0 mg), glabrol (6.0 mg), and 4′-O-methylglabridin (5.2 mg). The amount of each component was measured using a calibration curve prepared from the result of an HPLC analysis of a commercially available standard substance containing a known concentration of the component.

### Polyphenol Analysis

The polyphenol content was measured by a Folin-Denis method using glabridin (a commercially available reagent) as a standard substance, with the result that the total amount of polyphenol contained in 1 g of the medium-chain fatty acid triglyceride solution containing a hydrophobic licorice extract was 239.1 mg.

### HPLC Conditions for Glycyrrhizic Acid Analysis

Column: YMC J'sphere ODS-H80, 4.6 mm in inner diameter×250 mm in length (YMC Co., Ltd.)

Column temperature: 40° C.

Mobile phase A: acetonitrile

Mobile phase B: 20 mM phosphoric acid water solution

Gradient: the ratio of the mobile phase A to the total amount of the mobile phase A and the mobile phase B was constant at 36% after the start of analysis until 10 minutes after the start, then increased to 45% at a constant ratio after the 10 minutes until 50 minutes, then constant at 100% after the 50 minutes until 55 minutes, and constant at 36% after the 55 minutes until 75 minutes.

Flow rate: 1 mL/min

Wavelength: UV 254 nm

Amount of sample injected: 20 μL

### Glycyrrhizic Acid Analysis

The amount of the glycyrrhizic acid contained in the medium-chain fatty acid triglyceride solution containing a hydrophobic licorice extract in Example 2 was measured under the above-mentioned conditions, and found to be equal to or less than the detection limit (0.001 wt %).

### Example 3

Medium-chain fatty acid triglyceride solutions 1 to 8 containing a hydrophobic licorice extract were obtained by the same methods as in Examples 1 and 2 except that the rhizome portions of different licorices harvested in different places at different periods of time were used as raw materials. The peak intensities measured by HPLC from the glabridin, glabrene, glabrol, and 4′-O-methylglabridin contained in the triglyceride solutions 1 to 8 obtained are as in the following Table.

TABLE 1

| | Peak Intensity (%, to Glabridin) | | | |
|---|---|---|---|---|
| Solution | Glabrene | Glabridin | Glabrol | 4′-O-Methylglabridin |
| 1 | 39.0 | 100.0 | 46.3 | 18.8 |
| 2 | 39.0 | 100.0 | 46.4 | 18.8 |
| 3 | 38.9 | 100.0 | 46.2 | 18.8 |
| 4 | 38.9 | 100.0 | 45.9 | 18.7 |
| 5 | 39.0 | 100.0 | 46.3 | 18.9 |
| 6 | 39.0 | 100.0 | 46.4 | 18.9 |
| 7 | 41.3 | 100.0 | 43.8 | 16.7 |
| 8 | 40.7 | 100.0 | 46.1 | 15.5 |

### Example 4

### Test Example 1—Stabilization of Transthyretin Tetramer

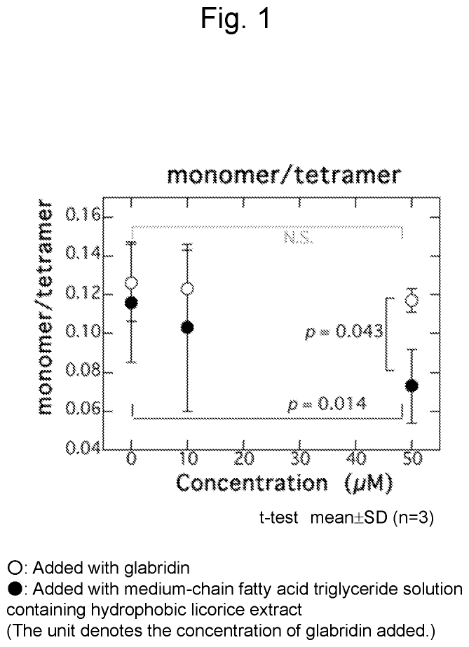

Serums were obtained from blood taken from three middle-aged or older persons. The medium-chain fatty acid triglyceride solution containing a hydrophobic licorice extract and the glabridin that were obtained in Example 2 were added to each serum (added in such a manner that the amount of the glabridin was 0, 10, and 50 μM respectively). The resulting mixtures were incubated at 25° C. for 30 minutes, and then denatured with urea. Using these samples, the transthyretins (the tetramers and the monomers) in the blood were quantitated by electrophoresis and Western blotting. The stabilization effect was investigated using the ratio of the monomers to the tetramers (monomer/tetramer). The result is shown in FIG. 1.

As shown in FIG. 1, the ratio of the monomers to the tetramers did not change with the glabridin added at the concentrations up to 50 μM, and the stabilization effect of the transthyretin was not observed. On the other hand, the ratio of the monomers to the tetramers was decreased when the concentration of the medium-chain fatty acid triglyceride solution containing a hydrophobic licorice extract was the largest. Specifically, the effect of decreasing the ratio of the monomers and increasing the tetramers (the stabilization of transthyretin) was observed.

### Test Example 2—Stabilization of Transthyretin Tetramer

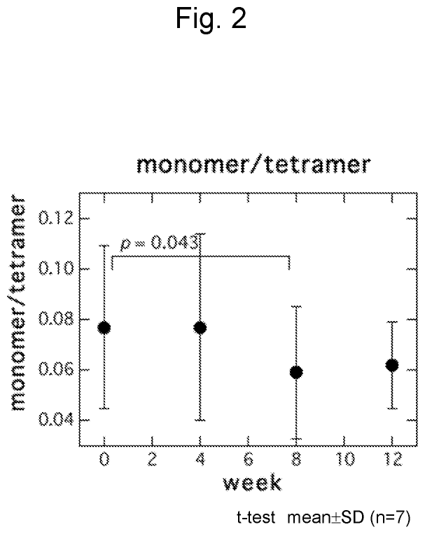

Over a period of 12 weeks, seven middle-aged or older persons took in that medium-chain fatty acid triglyceride solution containing a hydrophobic licorice extract which was obtained in Example 2 (the amount of ingestion, 300 mg/day). Blood was collected at time intervals, whereby plasma was obtained. The plasma of each person was incubated at 25° C. for 30 minutes, and then, denatured with urea. Using these samples, the transthyretins (the tetramers and the monomers) in the blood were quantitated by electrophoresis and Western blotting. The stabilization effect was investigated using the ratio of the monomers to the tetramers (monomer/tetramer). The result is shown in FIG. 2.

As shown in FIG. 2, the ratio of the monomers to the tetramers was decreased after 8 weeks of the supplementation with the medium-chain fatty acid triglyceride solution containing the hydrophobic licorice extract. Specifically, the effect of decreasing the ratio of the monomers and increasing the tetramers (the stabilization of transthyretin) was found.

As above-mentioned, the hydrophobic licorice extract can stabilize the transthyretin tetramer, decrease the ratio of the monomers contained in vivo, and increase the tetramer.

All the publications, patents, and patent applications cited herein are incorporated herein by reference in their entireties.

1. A transthyretin tetramer stabilizing agent comprising a hydrophobic licorice extract containing a licorice glabra polyphenol.

2. The agent according to claim 1, wherein the licorice glabra polyphenol contains at least glabrene, glabridin, glabrol, and 4′-O-methylglabridin.

3. The agent according to claim 1, wherein the hydrophobic licorice extract exhibits any one or more of the characteristics b), c), and d) under the following conditions a) in an HPLC analysis:
a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;
b) a ratio of a glabrene peak intensity to a glabridin peak intensity is 38% or more and 41% or less;
c) a ratio of a glabrol peak intensity to a glabridin peak intensity is 44% or more and 47% or less; and
d) a ratio of a 4′-O-methylglabridin intensity to a glabridin peak intensity is 15% or more and 20% or less.

4. The agent according to claim 1, further comprising, in addition to the hydrophobic licorice extract, one or more members selected from the group consisting of glabrene, glabridin, glabrol, or 4′-O-methylglabridin.

5. The agent according to claim 1, wherein the content of glycyrrhizic acid is 0.005 wt % or less.

6. A treatment agent, preventing agent or progression suppressing agent for transthyretin amyloidosis comprising a hydrophobic licorice extract containing a licorice glabra polyphenol.

7. The agent according to claim 6, wherein the transthyretin amyloidosis is senile systemic amyloidosis or familial amyloid polyneuropathy.

8. The agent according to claim 6, wherein the licorice glabra polyphenol contains at least glabrene, glabridin, glabrol, and 4′-O-methylglabridin.

9. The agent according to claim 6, wherein the hydrophobic licorice extract exhibits any one or more of the characteristics b), c), and d) under the following conditions a) in an HPLC analysis:
a) mobile phase: acetonitrile:methanol=1:1 (mobile phase A) and 20 mM phosphoric acid (mobile phase B) in gradient elution; column: ODS column; flow rate: 1.0 mL/minute; temperature: 40° C.; detector: UV detector; and wavelength of detection: 282 nm;
b) a ratio of a glabrene peak intensity to a glabridin peak intensity is 38% or more and 41% or less;
c) a ratio of a glabrol peak intensity to a glabridin peak intensity is 44% or more and 47% or less; and
d) a ratio of a 4′-O-methylglabridin intensity to a glabridin peak intensity is 15% or more and 20% or less.

10. The agent according to claim 6, further comprising, in addition to the hydrophobic licorice extract, any one or more selected from the group consisting of glabrene, glabridin, glabrol, or 4′-O-methylglabridin.

11. The agent according to claim 1, in the form of a tablet, a capsule, a granule, or a powder.

12. The agent according to claim 1, comprising glabridin in an amount of 4 mg to 1200 mg per one ingestion or dosage unit.

13. A method of treating, preventing, or suppressing the progression of transthyretin amyloidosis in a subject, comprising:
administering an effective amount of a hydrophobic licorice extract comprising a licorice glabra polyphenol to a subject in need of the prevention of, or the suppression of progression of, transthyretin amyloidosis; and
suppressing, in the subject, the monomerization of a transthyretin tetramer and/or the formation of amyloid fibrils derived from the transthyretin tetramer.

14. A pharmaceutical composition comprising a hydrophobic licorice extract containing a licorice glabra polyphenol and a pharmaceutically acceptable carrier.

15. The pharmaceutical composition according to claim 14 which is in the form of a tablet, a capsule, a granule, or a powder.

16. The pharmaceutical composition according to claim 14 comprising glabridin in an amount of 4 mg to 1200 mg per dosage.

17. A method of treating senile systemic amyloidosis or familial amyloid polyneuropathy comprising administering to a subject in need of such treatment an effective amount of a hydrophobic licorice extract comprising a licorice glabra polyphenol.

18. The method according to claim 17, wherein the licorice extract is administered in an amount of 0.01 to 100 mg/kg of body weight per day.
